(19) United States
(12) Patent Application Publication
(10) Pub. No.: US 2017/0056229 A1
(43) Pub. Date: Mar. 2, 2017

(54) SYSTEMS FOR CORRECTING DIGITAL DEFORMITIES

(71) Applicant: MX Orthopedics, Corp., Lexington, MA (US)

(72) Inventors: Matthew Palmer, Cambridge, MA (US); Matthew Fonte, Concord, MA (US); Robert Devaney, Auburndale, MA (US)

(21) Appl. No.: 15/255,518

(22) Filed: Sep. 2, 2016

Related U.S. Application Data
(60) Provisional application No. 62/213,142, filed on Sep. 2, 2015.

(51) Int. Cl. A61F 5/01

(52) U.S. Cl. CPC A61F 5/019; A61F 5/0127

(57) ABSTRACT

This disclosure provides a novel superelastic nickel-titanium (Nitinol or NiTi) splint/orthotic that maximizes the ability to slowly correct toe deformities by utilizing the viscoelastic properties of the bone. The superelastic Nitinol digital deformity orthotic is safe, can gradually correct digit curves, and ultimately results in better toe alignments compared to contemporary static splints or surgery.

### CROSS-REFERENCE TO RELATED APPLICATIONS

This application claims priority to U.S. Provisional Application No. 62/213,142, filed on Sep. 2, 2015, the disclosure of which is incorporated herein by reference in its entirety.

### TECHNICAL FIELD

The present disclosure relates to systems for non-surgically correcting deformities of the digits. The disclosure finds particular utility in the field of orthopedics and specifically for non-surgically correcting deformities of the digits by applying and maintaining a force to counter the direction of the deformity. While the disclosure has applications throughout the body, its utility will be illustrated herein in the context of the repair of injured bone tissue, and specifically to correct hammertoes, bunions, and other deformities of the digits.

### BACKGROUND

In the field of orthopedics it is common to treat deformities of the digits. Deformities often result from shoes that do not fit properly. Alternatively, deformities may be generated by muscle imbalance. Muscles work in pairs to straighten and bend the toes. If the toe is bent and held in one position long enough, the muscles tighten and cannot stretch out.

Shoes that narrow toward the toe may push the smaller toes into a flexed (bent) position. The toes rub against the shoe, leading to the formation of corns and calluses. A higher heel forces the foot down and squishes the toes against the shoe, increasing the pressure and the bend in the toe. Eventually, the toe muscles become unable to straighten the toe, even when there is no confining shoe.

One of the most common deformities is called a hammertoe which is characterized by an extension deformity at the metatarsophalangeal joint (MPJ) and a flexion deformity at the proximal interphalangeal joint.

Digital deformities typically are associated with pain in the forefoot, difficulty fitting shoes and an unacceptable cosmetic appearance for the affected patients. Forefoot pain is typically attributed to hyperkeratotic skin lesions, callosities or ulcerations about the MPJ, the proximal interphalangeal joint and distal interphalangeal joints as well as nail deformities secondary to the curling of the digits. Overloading the MPJ as well as the surrounding digits ultimately changing the gait pattern. This often advances other forefoot etiologies.

Physicians have several treatment options ranging from conservative to surgical. Conservative treatment starts with shoes that are soft and roomy. Shoes should be one-half inch longer than the longest toe. Tight, narrow, high-heeled shoes should be avoided. Physicians may prescribe toe exercises that stretch and strengthen the muscles. Physicians may recommend the use of commercially available straps, cushions, toe sleeves, foam cushioning devices, orthotics or offloading pads such as a metatarsal bar to relieve symptoms.

If conservative measures fail, physicians may recommend surgery. Usually, surgery is done on an outpatient basis with a local anesthetic. The actual procedure will depend on the type and extent of the deformity but usually includes joint resection. In this procedure an incision is made over the top of the toe. Ligaments and tendons are cut to help with straightening the toe. The distal face of the bone is removed to allow the toe to straighten completely, and an implant is used to hold the toe straight. The implant may be removed three to four weeks after the surgery.

While surgery often successfully treats digital deformities, it is possible that the deformity may return and require repeat surgery. Despite the myriad of static orthotic instrumentation systems, deformities can rarely be fully corrected, especially when the curves are stiff and do not contour to the bone. Thus there exists a clinical need for systems to slowly, non-surgically correct digit deformities by utilizing the viscoelastic properties of the bone.

### SUMMARY

The present disclosure provides a novel superelastic nickel-titanium (Nitinol or NiTi) splint/orthotic that maximizes the ability to slowly correct toe deformities by utilizing the viscoelastic properties of the bone. The superelastic Nitinol digital deformity orthotic is safe, can gradually correct digit curves, and ultimately results in better toe alignments compared to contemporary static splints or surgery.

Among other things, the present disclosure comprises the provision and use of a splint that incorporates a shape memory material (e.g., a material capable of exhibiting superelasticity and/or a temperature-induced shape change). The shape memory material may be a metal alloy (e.g., Nitinol) or a polymer (e.g., appropriately processed PEEK). The splint is designed to generate and maintain a force that counteracts the direction of the bone deformity.

The splint may be incorporated into an orthotic or into a shoe.

### BRIEF DESCRIPTION OF THE DRAWINGS

The various features and advantages of this disclosure will become apparent to those skilled in the art from the following detailed description. The drawings that accompany the detailed description can be briefly described as follows.

FIG. 1 is a schematic showing the hysteresis curve for the loading and unloading of a Nitinol member.

FIG. 2 is a schematic of a normal foot.

FIGS. 3-6 are schematics of different digital deformities of a foot.

FIG. 7 is a schematic of a Nitinol element that may be part of a splint.

FIG. 8 is a schematic of different splints that can incorporate a Nitinol element to slowly correct the digital deformity.

### DETAILED DESCRIPTION

A system for correcting a bone deformity according to an exemplary aspect of this disclosure includes, inter alia, a deformity correcting orthotic that includes a shape memory material configured to generate and maintain a first force that counteracts a second, opposing force of a bone deformity.

In a further embodiment, a shape memory material is a metal alloy.

In a further embodiment, a metal alloy is Nitinol.

In a further embodiment, a shape memory material is a polymer.

In a further embodiment, a polymer is a PEEK material.

In a further embodiment, a deformity correcting orthotic is a splint or brace.

In a further embodiment, a deformity correcting orthotic is a stent.

In a further embodiment, a deformity correcting orthotic is padded with cloth.

In a further embodiment, a deformity correcting orthotic is fitted into a sock.

In a further embodiment, a deformity correcting orthotic is fitted into a shoe.

A method for correcting a bone deformity according to another exemplary aspect of this disclosure includes, inter alia, positioning a deformity correcting orthotic in proximity to a bone deformity of a bone, and maintaining a constant force against the bone deformity with the deformity correcting orthotic to gradually realign the bone. The deformity correcting orthotic includes a shape memory material.

In a further embodiment, a shape memory material is a self-shape changing Nitinol configured to revert to a pre-defined shape.

In a further embodiment, a deformity correcting orthotic is a splint and positioning the deformity correcting orthotic includes positioning the splint directly against a bone such that a constant force counteracts an opposing force of a bone deformity.

In a further embodiment, a deformity correcting orthotic is a stent and positioning the deformity correcting orthotic includes slipping the stent over a bone such that a constant force counteracts an opposing force of a bone deformity.

In a further embodiment, a deformity correcting orthotic is padded with cloth.

In a further embodiment, a deformity correcting orthotic is fitted into a sock.

In a further embodiment, a deformity correcting orthotic is fitted into a shoe.

In a further embodiment, a deformity correcting orthotic is positioned in proximity to a finger or toe bone.

In a further embodiment, a constant force is generated by a shape memory material of a deformity correcting orthotic as it attempts to revert to its pre-defined shape.

Remodeling (reshaping) of a bone is the process by which bones change their overall shape in response to physiologic influences or mechanical forces, leading to gradual adjustment of the skeleton to the forces that it encounters. Bones may widen or change axis by removal or addition of bone to the appropriate surfaces by independent action of osteoblasts and osteoclasts in response to biomechanical forces. Bones normally widen with aging in response to periosteal apposition of new bone and endosteal resorption of old bone. Wolff's law describes the observation that long bones change shape to accommodate stresses placed on them. Remodeling is regulated by mechanical loading, allowing bone to adapt its structure in response to the mechanical demands Physical activity is essential for the correct development of bone. It is believed that muscular action transmits tension to the bone, which is detected by the osteocyte network within the osseous fluid. On the other hand, the absence of muscular activity, rest, or weightlessness has an adverse effect on bone, accelerating resorption. It is well-known that trabeculae tend to align with maximum stresses in many bones. Mechanical stress improves bone strength by influencing collagen alignment as new bone is being formed. Cortical bone tissue located in regions subject to predominantly tensile stresses has a higher percentage of collagen fibers aligned along the bone long axis. In regions of predominant compressive stresses, fibers are more likely to be aligned transverse to the long axis. A stent or splint that can apply stress to a bone will cause the bone to remodel and change and realign its shape over time.

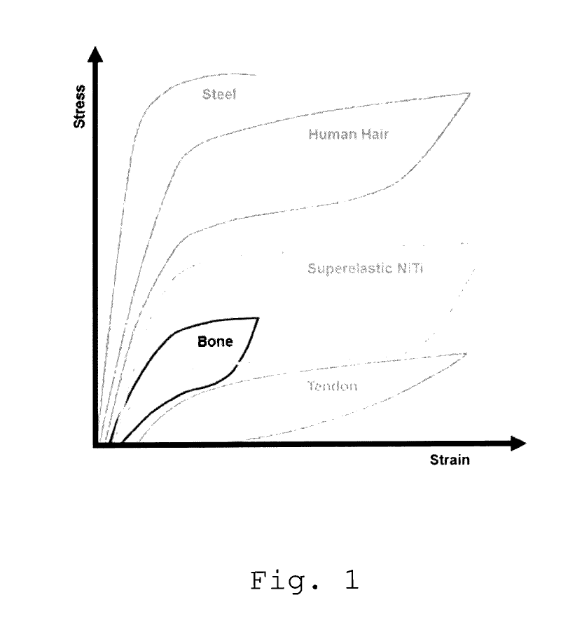

As seen in FIG. 1, Nitinol (NiTi) follows the same stress strain hysteresis as bone, making it a very natural material to be used in conjunction with bone. Nitinol can be 10 times more elastic compared to the stainless steels used in the medical field today and follows a non-linear path, characterized by a pronounced hysteresis. While in most engineering materials stress increases with strain upon loading in a linear way and decreases along the same path upon unloading, Nitinol exhibits a distinctly different behavior. Upon loading, stress first increases linearly with strain, up to 1% strain. After a first ‘yield point’, several percentage points of strain can be accumulated with only a little stress increase. The end of this plateau (‘loading plateau’) is reached at about 8% strain. After that point, there is another, second, linear increase of stress with strain. Unloading from the end of the plateau region causes the stress to decrease rapidly, until a lower plateau (‘unloading plateau’) is reached. Strain is recovered in this region with only a small decrease in stress. The last portion of the deforming strain is finally recovered in a linear fashion again. The unloading stress can be as low as 25% of the loading stress. The looped stress/strain hysteresis of Nitinol is similar to the hysteresis exhibited by bone, making the two materials compatible.

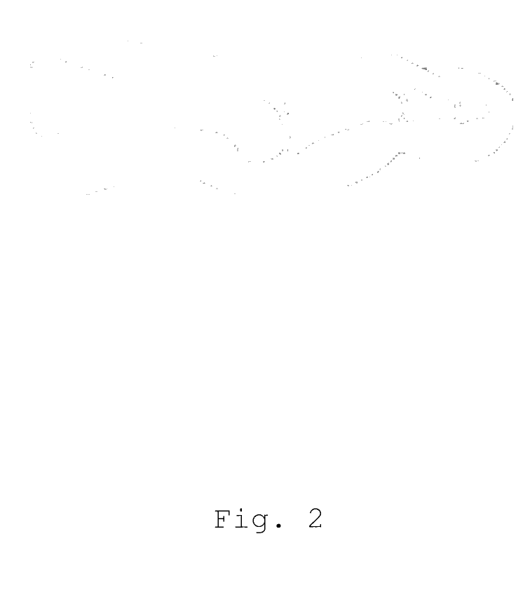

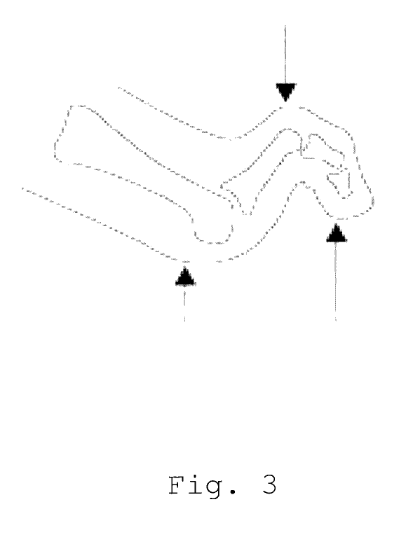

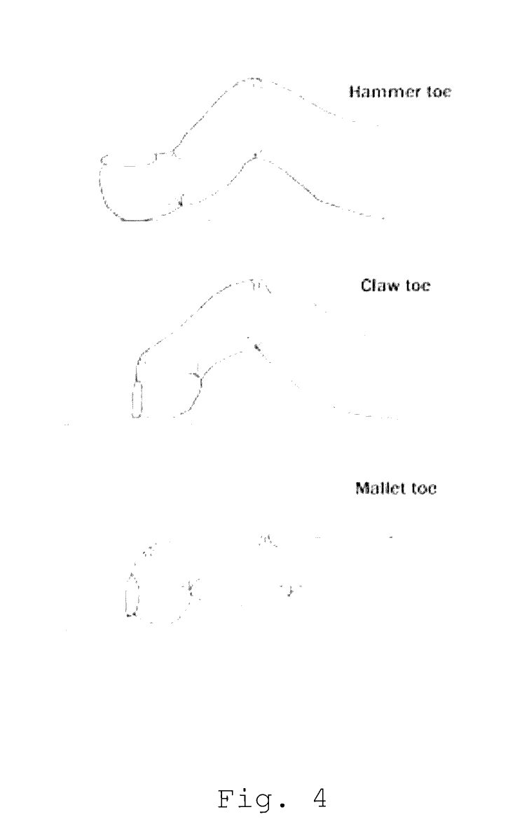

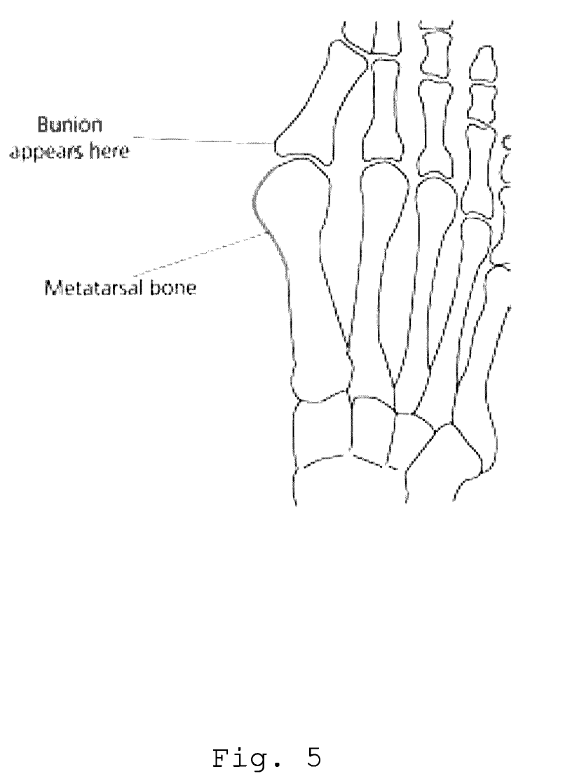

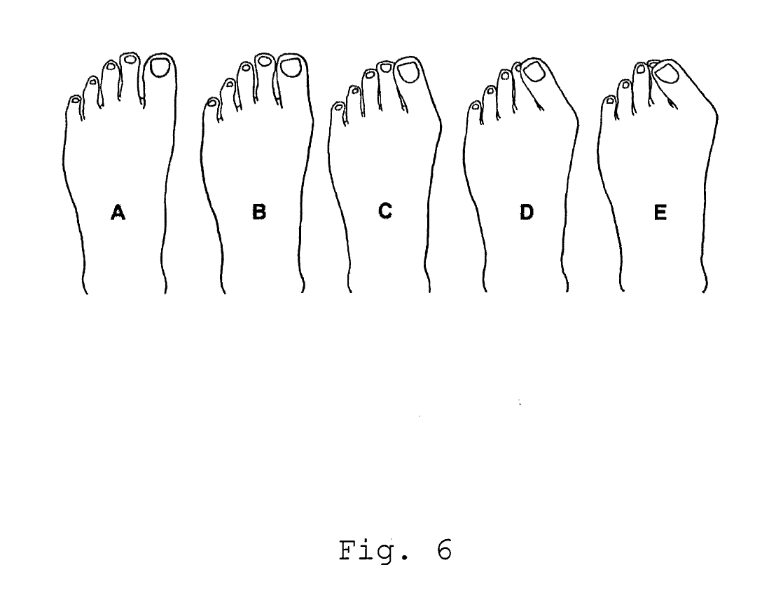

As previously stated, digital deformities may be caused by the wearing of tight, misfitting shoes and/or may be caused by muscle imbalance. FIG. 2 shows a normal foot, with the bones properly aligned. FIG. 3 shows an abnormal toe with deformities caused by pressure points. If untreated, the abnormality may progress to a hammertoe, claw-toe or mallet-toe, as shown in FIG. 4. Bunions are another deformity of the first metatarsal. As seen in FIGS. 5-6, when not corrected, the bunion will cause the first phalange to bend and, in severe cases, to cross over the phalange of the second metatarsal.

Nitinol can be incorporated into splints and braces to generate and maintain a force to stimulate bone remodeling in order to alleviate imbalances that cause the deformity. Additionally, the Nitinol element has the following benefits:
Elastic deployment: The enormous elasticity of Nitinol allows devices to be constrained in one condition and then released from the constraint to put stress on bone to help realign naturally.
Constant stress: An important feature of superelastic Nitinol alloys is that its loading and unloading curves are substantially flat over large strains. This allows the design of devices that apply a constant stress over a wide range of shapes. The NiTi splint device will offer a constant stress under loading or unloading conditions, i.e. laying down or walking.
Dynamic interference: Dynamic interference refers to the long-range nature of Nitinol stresses and can be clearly illustrated using NiTi splints. Unlike static non-NiTi splints, self-shape changing Nitinol splints can be developed to always try to revert to their pre-set shape. The Nitinol toe device will continue to gently push outward against the toe, restoring it's curvature to a more normal alignment, thus improving motion and eliminating pain.

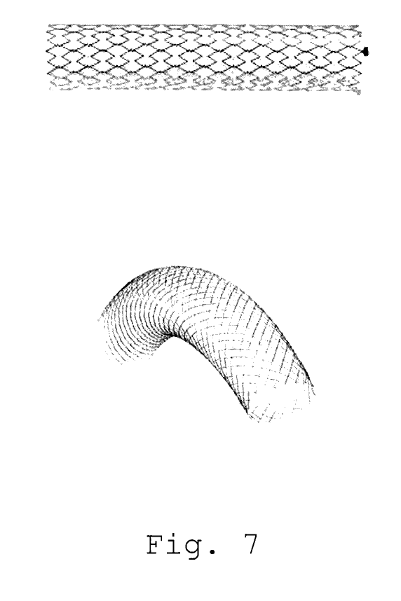

FIG. 7 shows one embodiment of the present disclosure. It is possible to create a NiTi stent and slip it over the hammer toe. The NiTi stent will have been heat treated (aged) to have a superelastic bias in the opposite direction of the hammer toe deformity. As the NiTi stent applies strain against the curved, deformed toe, stress is generated and applied in a controlled manner against the toe. This chronic stress will re-align the toe to its original, normal position.

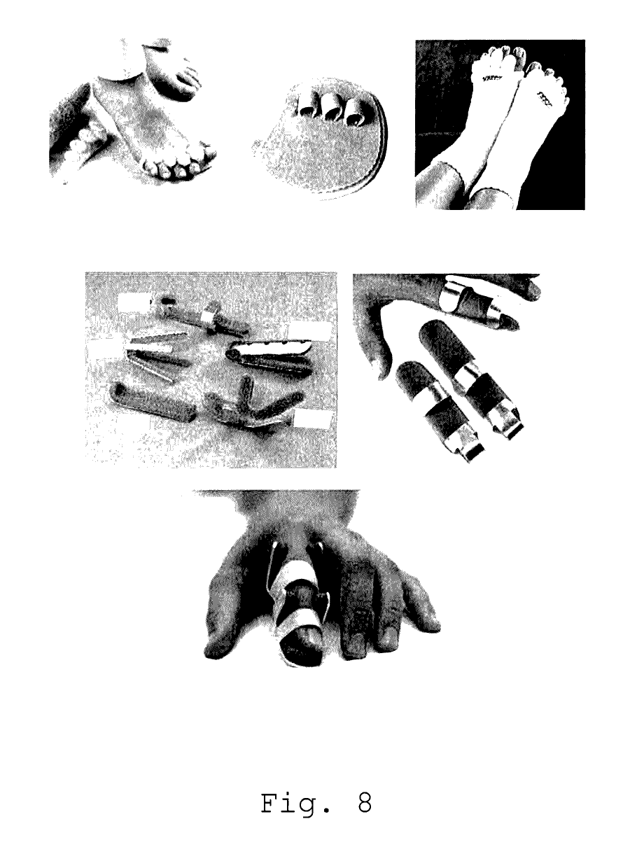

As seen in FIG. 8, these NiTi stents can be padded with cloth for improved comfort or fitted into a comfortable toe sock. The severity of the curvature of the NiTi stents can be modified over time by subsequent heat treatments; alternatively, a series of splints can be employed with increasing degrees of curvature biases resulting in a superelastic NiTi hammer toe corrective system. The devices can be circular like a stent or could be more like a traditional finger splint but made of NiTi and used on the toe.

This disclosure provides a novel superelastic nickel-titanium (Nitinol or NiTi) splint/orthotic that maximizes the ability to slowly correct toe deformities by utilizing the viscoelastic properties of the bone. The superelastic Nitinol digital deformity orthotic is safe, can gradually correct digit curves, and ultimately results in better toe alignments compared to contemporary static splints or surgery.

The foregoing description shall be interpreted as illustrative and not in any limiting sense. A worker of ordinary skill in the art would recognize that certain modifications could come within the scope of this disclosure. For these reasons, the following claims should be studied to determine the true scope and content of this disclosure.

What is claimed is:

1. A system for correcting a bone deformity, comprising:
a deformity correcting orthotic that includes a shape memory material configured to generate and maintain a first force that counteracts a second, opposing force of a bone deformity.

2. The system as recited in claim 1, wherein the shape memory material is a metal alloy.

3. The system as recited in claim 2, wherein the metal alloy is Nitinol.

4. The system as recited in claim 1, wherein the shape memory material is a polymer.

5. The system as recited in claim 4, wherein the polymer is a PEEK material.

6. The system as recited in claim 1, wherein the deformity correcting orthotic is a splint or brace.

7. The system as recited in claim 1, wherein the deformity correcting orthotic is a stent.

8. The system as recited in claim 1, wherein the deformity correcting orthotic is padded with cloth.

9. The system as recited in claim 1, wherein the deformity correcting orthotic is fitted into a sock.

10. The system as recited in claim 1, wherein the deformity correcting orthotic is fitted into a shoe.

11. A method for correcting a bone deformity, comprising:
positioning a deformity correcting orthotic in proximity to a bone deformity of a bone, the deformity correcting orthotic including a shape memory material; and
maintaining a constant force against the bone deformity with the deformity correcting orthotic to gradually realign the bone.

12. The method as recited in claim 11, wherein the shape memory material is a self-shape changing Nitinol configured to revert to a pre-defined shape.

13. The method as recited in claim 11, wherein the deformity correcting orthotic is a splint and the positioning includes:
positioning the splint directly against the bone such that the constant force counteracts an opposing force of the bone deformity.

14. The method as recited in claim 11, wherein the deformity correcting orthotic is a stent and the positioning includes:
slipping the stent over the bone such that the constant force counteracts an opposing force of the bone deformity.

15. The method as recited in claim 11, comprising, prior to the positioning:
padding the deformity correcting orthotic with cloth.

16. The method as recited in claim 11, comprising, prior to the positioning:
fitting the deformity correcting orthotic into a sock.

17. The method as recited in claim 11, comprising, prior to the positioning:
fitting the deformity correcting orthotic into a shoe.

18. The method as recited in claim 11, wherein the bone is part of a finger or a toe.

19. The method as recited in claim 11, wherein the constant force is generated by the shape memory material when attempting to revert to its pre-defined shape.
